(19) United States
(12) Patent Application Publication
(10) Pub. No.: US 2017/0014057 A1
(43) Pub. Date: Jan. 19, 2017

(54) BIOLOGICAL-INFORMATION MEASUREMENT DEVICE

(71) Applicant: Masahiro SATO, Tokyo (JP)

(72) Inventor: Masahiro SATO, Tokyo (JP)

(73) Assignee: MITSUMI ELECTRIC CO., LTD., Tokyo (JP)

(21) Appl. No.: 15/107,242

(22) PCT Filed: Dec. 17, 2014
(86) PCT No.: PCT/JP2014/006288

§ 371 Date: Jun. 22, 2016

(30) Foreign Application Priority Data
Dec. 27, 2013 (JP) 2013-272964

(51) Int. Cl. A61B 5/145; A61B 5/1455; A61B 5/1495

(52) U.S. Cl. CPC A61B 5/14542; A61B 5/1495; A61B 5/1455; A61B 2562/028; A61B 2562/0242

(57) ABSTRACT

Provided is a biological-information measurement device having a small-sized device configuration without a reduction in measurement precision. Reflected light from a measurement object (10) is split using a rotary diffraction grating (110), and the space requirements and number of components of a light-splitting optical system can thereby be reduced. A reflecting member (140) for reflecting light incident from a measurement probe (106) instead of the measurement object (10) and emitting the light to the measurement probe (106) is also provided, whereby an optical path for obtaining a measurement signal and an optical path for obtaining a reference signal can be configured in common, space requirements can be reduced, and precision of calibration can be enhanced. As a result, particularly the light-splitting optical system and a reference signal optical system can be reduced in size without a reduction in measurement precision.

### TECHNICAL FIELD

The present invention relates to a biological information measurement device which noninvasively measures biological information such as blood glucose level with use of light.

### BACKGROUND ART

Conventionally, apparatuses which irradiate a sample (human body) with a near infrared ray and analyze the reflection light from the sample to noninvasively measure the blood glucose level. Such apparatuses are disclosed in PTLS 1 to 4 for example.

In general, such apparatuses include: a first optical system configured to guide light from a light source to a measurement target; a second optical system configured to guide light reflected by the measurement target; an optical system configured to split the reflection light guided by the second optical system; a photodetector configured to receive the split light; and a reference signal optical system configured to generate a reference signal for calibration.

### CITATION LIST

### Patent Literatures

PTL 1

Japanese Patent Application Laid-Open No. 2006-87913

PTL 2

Japanese Patent Application Laid-Open No. 2002-65465

PTL 3

Japanese Patent Application Laid-Open No. 2007-259967

PTL 4

Japanese Patent Application Laid-Open No. 2012-191969

### SUMMARY OF INVENTION

### Technical Problem

Incidentally, when the above-described biological information measurement device is downsized so that the detector can be carried, the user can measure the blood glucose level at any time, which is very convenient. In addition, advantageously, the biological information measurement device can be easily incorporated in other health management devices such as a conventional body composition meter when the biological information measurement device is downsized.

However, since the photodetector which is a principal component is composed of an array type sensor in the above-described conventional biological information measurement devices, the above-described conventional biological information measurement devices are still insufficient in terms of downsizing.

In consideration of the above-mentioned points, the present invention provides a biological information measurement device which can achieve downsizing of the device configuration without reducing the measurement accuracy.

### Solution to Problem

A biological information measurement device according to a mode of the present invention includes: a light source; a first optical path configured to guide light emitted from the light source to a measurement target; a second optical path configured to guide reflection light reflected from the measurement target; a rotation diffraction grid configured to split reflection light guided from the second optical path; a light receiving element configured to receive split light from the rotation diffraction grid; and a reflection member configured to reflect incident light from the first optical path and emit the incident light to the second optical path in place of the measurement target.

### Advantageous Effects of Invention

According to the present invention, a biological information measurement device which can achieve downsizing of the device configuration without reducing the measurement accuracy can be provided.

### BRIEF DESCRIPTION OF DRAWINGS

FIG. 1 is a schematic view illustrating a general configuration of a biological information measurement device according to the embodiment;

FIG. 2A to FIG. 2C illustrate a diffraction operation of a rotation diffraction grid;

FIG. 3 is a plan view illustrating an external configuration of a MEMS device provided with the rotation diffraction grid;

FIG. 4A and FIG. 4B illustrate variation of the intensity of a signal measured by a photo detector (PD) in the case where the position of the rotation diffraction grid is changed in a direction perpendicular to a mirror surface with the same rotation position of the rotation diffraction grid;

FIG. 5A to FIG. 5D are graphs for describing lock-in amplifier detection;

FIG. 6A and FIG. 6B illustrate movement of a reflection member; and

FIG. 7A and FIG. 7B are sectional views illustrating an exemplary configuration of the reflection member.

### DESCRIPTION OF EMBODIMENT

In the following, an embodiment of the present invention is described in detail with reference to the accompanying drawings.

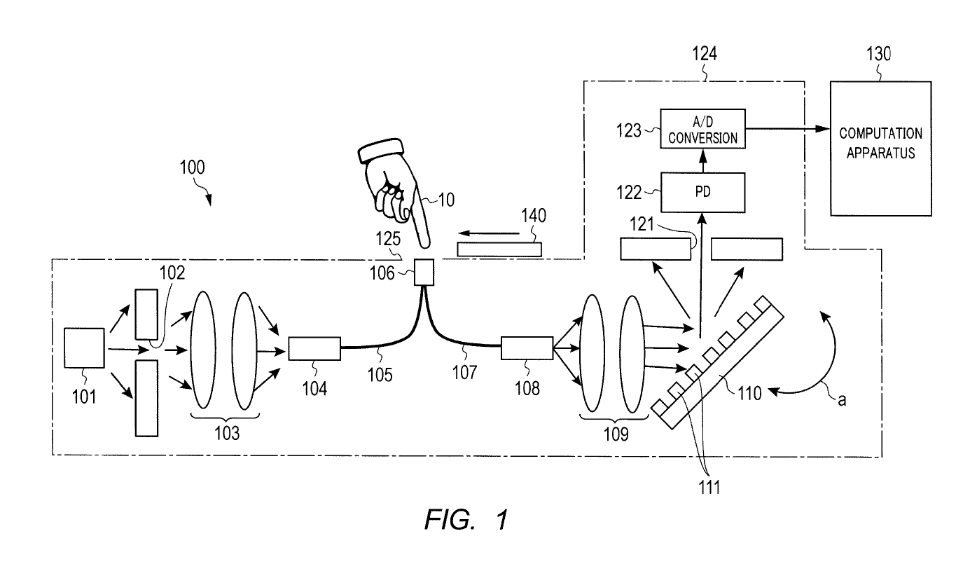

FIG. 1 is a schematic view illustrating a general configuration of a biological information measurement device according to an embodiment of the present invention. Biological information measurement device 100 irradiates subject 10 as a measurement target with near-infrared light and analyzes the reflection light to noninvasively measure the blood glucose level of subject 10 as biological information.

Biological information measurement device 100 generates a near infrared ray by light source 101. Light source 101 is composed of an LED (Light Emitting Diode), and a halogen lamp or a xenon lamp. The light from light source 101 passes through pin hole 102 and is then condensed by condenser lens 103. The condensed light is incident on light emission side optical fiber 105 from light incidence body 104. One end of light emission side optical fiber 105 is connected with light incidence body 104, and the other end of light emission side optical fiber 105 is connected with measurement probe 106. It is to be noted that pin hole 102 may not be provided.

Measurement probe 106 is provided at a position where the end of probe 106 can make contact with the surface of the skin of subject 10, or at a position where probe 106 can face the skin in the close vicinity of the skin. The near-infrared light applied to subject 10 through light emission side optical fiber 105 and measurement probe 106 enters the body of subject 10 and is then reflected, and thereafter, returns to measurement probe 106. The light returned to measurement probe 106 is emitted from light emission body 108 through light reception side optical fiber 107. The light emitted from light emission body 108 is converted into collimate light by lens system 109, and is then incident on rotation diffraction grid 110.

It is to be noted that, since the principle of measurement of biological information such as blood glucose level with use of near-infrared light is a publicly known technique, the detailed description thereof is omitted. In general, since the absorption intensity of near-infrared light in the body is largely influenced by the presence of glucose, the glucose density in the body, that is, the blood glucose level, is measured by measuring the absorption intensity.

Rotation diffraction grid 110 rotates as illustrated with arrow a in the drawing. The incidence surface of rotation diffraction grid 110 is a mirror surface, and reflects incident light. That is, rotation diffraction grid 110 rotates so as to change the incident angle to the mirror surface. The light reflected by rotation diffraction grid 110 passes through slit 121, and is then incident on photodetector (PD) 122. The light reception signal obtained through the photoelectric conversion by PD 122 is output to computation apparatus 130 through analog digital conversion circuit (A/D conversion) 123. Computation apparatus 130 is an apparatus such as a personal computer and a smartphone having an analysis program, and executes an analysis program to acquire biological information such as blood glucose level from the light reception signal.

It is to be noted that the optical system of biological information measurement device 100 is stored in case 124. In case 124, opening 125 for travelling of light between measurement probe 106 and subject 10 is formed at a position corresponding to measurement probe 106. It is to be noted that opening 125 may not be provided.

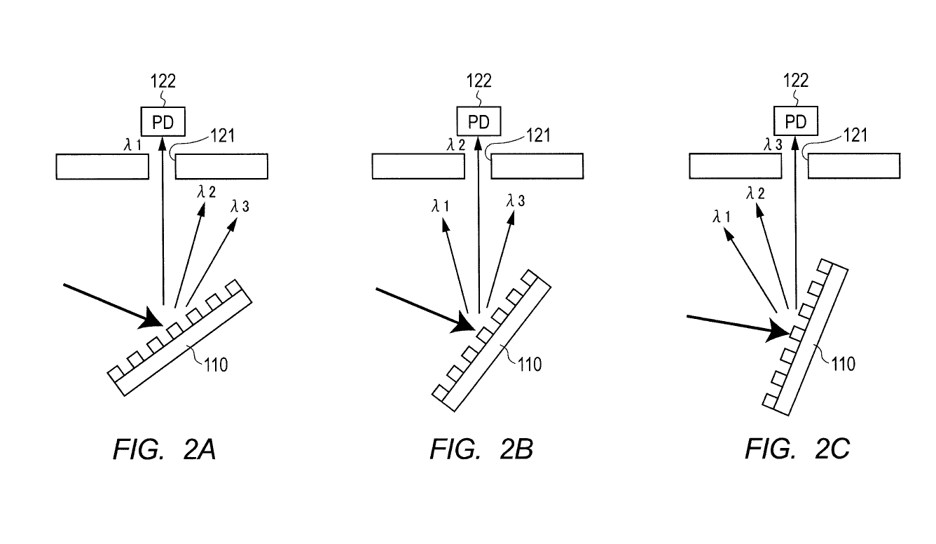

FIG. 2A to FIG. 2C illustrate a diffraction operation of rotation diffraction grid 110. When rotation diffraction grid 110 is located at a rotation position illustrated in FIG. 2A, rotation diffraction grid 110 reflects λ1 component of incident light in the direction of slit 121 so that λ1 component is incident on PD 122. In addition, when rotation diffraction grid 110 is located at a rotation position illustrated in FIG. 2B, rotation diffraction grid 110 reflects λ2 component of incident light in the direction of slit 121 so that λ2 component is incident on PD 122. Further, when rotation diffraction grid 110 is located at a rotation position illustrated in FIG. 2C, rotation diffraction grid 110 reflects λ3 component of incident light in the direction of slit 121 so that λ3 component is incident on PD 122. In this manner, rotation diffraction grid 110 inputs light of a wavelength corresponding to the rotation angle to PD 122 to thereby split the incident light.

In the present embodiment, since light is split using rotation diffraction grid 110, a photodetector composed of a single light reception surface, not an array sensor, can be used as photodetector (PD) 122 in comparison with the case of a fixed diffraction grid. Consequently, photodetector 122 having a simple configuration can be used, and as a result, cost can be reduced. In addition, in comparison with the case where a fixed diffraction grid is used, a space for splitting light between the diffraction grid and photodetector 122 is unnecessary, and thus the apparatus can be downsized.

Here, in rotation diffraction grid 110 of the present embodiment, the movable portion of a MEMS (Micro Electro Mechanical System) is a mirror surface, and a diffraction grid is formed on the mirror surface. That is, in rotation diffraction grid 110, a grating is formed on the mirror surface of the MEMS mirror.

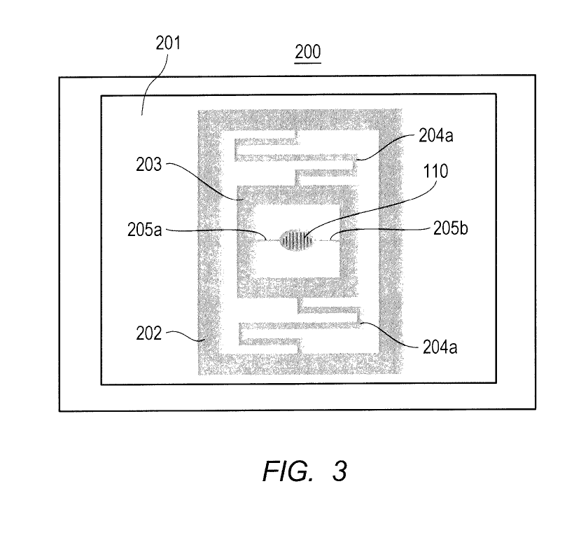

FIG. 3 is a plan view illustrating an external configuration of MEMS device 200 provided with rotation diffraction grid 110. MEMS device 200 includes driving part 201 composed of a driving circuit and an actuator; rotation diffraction grid 110; fixation frame 202; movable frame 203; and beam parts 204 and 205. Driving part 201 includes fixation frame 202 and has a function as a base of rotation diffraction grid 110 in addition to the function for driving rotation diffraction grid 110. Beam part 204 is composed of beams 204a and 204b. Beams 204a and 204b are provided as bridges between fixation frame 202 and opposite two edges of movable frame 203. Thus, movable frame 203 is suspended by fixation frame 202 with beams 204a and 204b. In addition, beam part 205 is composed of beams 205a and 205b. Beams 205a and 205b are provided as bridges between movable frame 203 and opposite two edges of rotation diffraction grid 110. Thus, rotation diffraction grid 110 is suspended by movable frame 203 with beams 205a and 205b.

Rotation diffraction grid 110 rotates when beams 204a and 204b are driven by driving part 201. To be more specific, when the left and right of beams 204a and 204b are changed by driving part 201 in a staggered manner in the depth direction of the drawing, rotation diffraction grid 110 is driven into rotation in a predetermined angle range. Rotation diffraction grid 110 is driven into rotation at a rotational speed of 1 to 2 [Hz]. It should be noted that the rotational speed is not limited to this example. The rotational speed may be selected in accordance with the arithmetic speed of computation apparatus 130. Examples of the system for driving rotation diffraction grid 110 include a piezoelectric system, an electrostatic system, and an electromagnetic driving system.

The surface of rotation diffraction grid 110 is a mirror surface, and further, diffraction grid 111 is formed on the mirror surface. Diffraction grid 111 is formed such that diffraction grid 111 is parallel to the axes of beams 204a and 204b. In the present embodiment, the pitch of diffraction grid 111 is 0.5 to 3 [μm]. In addition, the depth of diffraction grid 111 is 1.5 [μm] or greater. With this configuration, by rotation, rotation diffraction grid 110 can favorably split a near infrared ray. When using light rays other than the near infrared ray for measurement, it suffices to select the pitch and/or depth of diffraction grid 111 in accordance with the light ray.

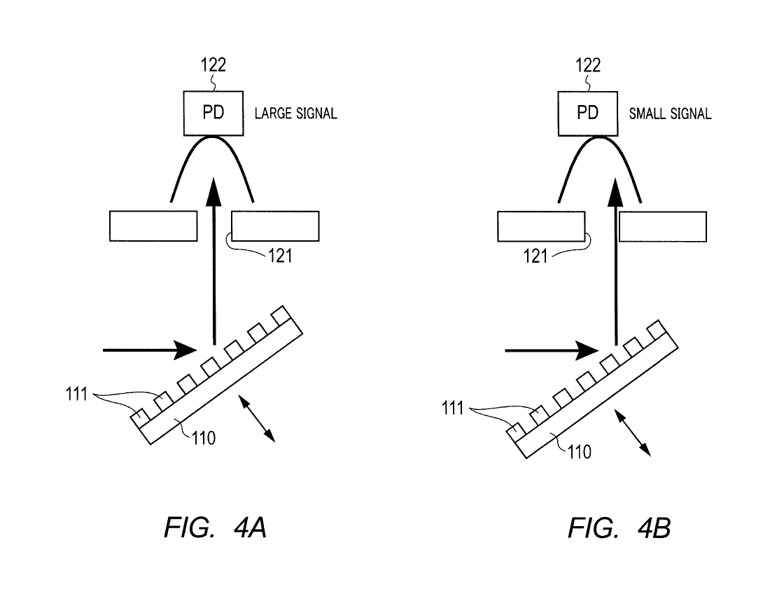

Further, in the present embodiment, rotation diffraction grid 110 is driven also in a direction perpendicular to the mirror surface as illustrated in FIG. 4A and FIG. 4B. To be more specific, beams 205a and 205b are simultaneously deflected by driving part 201 in the same depth direction of the drawing, and thus rotation diffraction grid 110 is driven in a direction perpendicular to the mirror surface. For example, a high-frequency simple harmonic motion of several tens of KHz is caused in a direction perpendicular to the mirror surface. FIG. 4A and FIG. 4B illustrate variation of the intensity of a signal measured by PD 122 in the case where the position of rotation diffraction grid 110 is changed in a direction perpendicular to the mirror surface with the same rotation position of rotation diffraction grid 110. Even when the rotation position is not changed, the amount of light passing through slit 121 is changed and the amount of light incident on PD 122 is changed as illustrated in FIG. 4A and FIG. 4B when the position in a direction perpendicular to the mirror surface is changed. With this configuration, a chopper signal can be superimposed on a measurement signal, and the noise component can be removed by performing lock-in amplifier detection. As a result, a signal with improved S/N can be obtained, and analysis accuracy is improved. It is to be noted that rotation diffraction grid 110 may be rotated by driving beams 205a and 205b. To be more specific, beams 205a and 205b are twisted in the same direction, and thus rotation diffraction grid 110 is driven into rotation in a predetermined angle range.

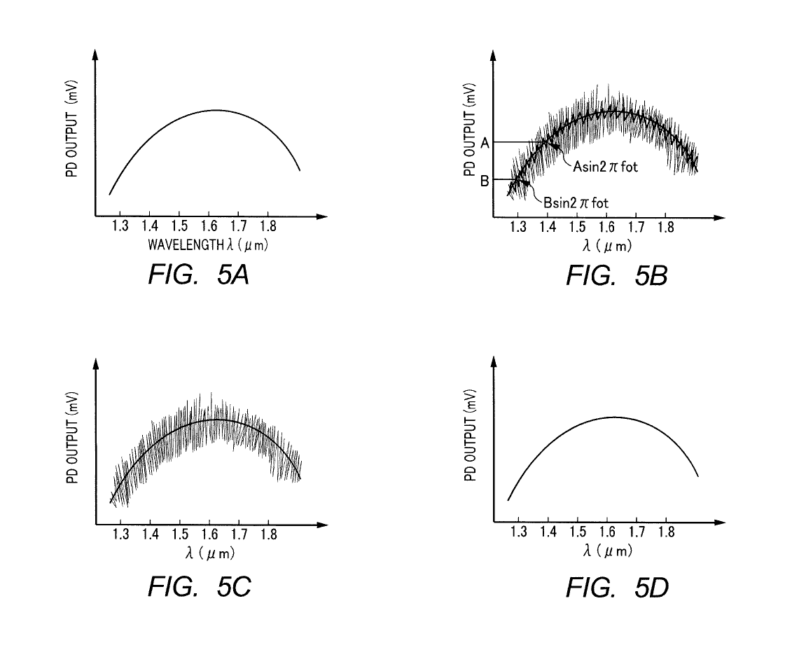

FIG. 5A to FIG. 5D are graphs for describing lock-in amplifier detection. FIG. 5A shows an ideal spectrum without noise. In actual measurement signals, noise of various frequencies are superimposed as shown in FIG. 5B. FIG. 5C shows a spectrum in the case where a high-frequency simple harmonic motion of frequency f₀ of rotation diffraction grid 110 is caused in a direction perpendicular to the mirror surface. As shown in FIG. 5C, a chopper signal having frequency f₀ is superimposed on the measurement signal. FIG. 5D shows a measurement signal subjected to lock-in amplifier detection. Only a signal of frequency f₀ can be extracted as a direct current signal (A and B in FIG. 5C). In this manner, the signals having frequencies other than f₀ are removed as noise.

As described above, in the present embodiment, rotation diffraction grid 110 is rotated to split measurement light, and a high-frequency simple harmonic motion of rotation diffraction grid 110 in a direction perpendicular to the mirror surface is caused to improve the S/N of the measurement signal. In other words, rotation diffraction grid 110 is biaxially driven in a rotational direction and a direction perpendicular to the mirror surface.

In addition to such configurations, biological information measurement device 100 of the present embodiment includes movable reflection member 140. Reflection member 140 is designed for obtaining a reference signal for calibration. As is well known, calibration is an operation for removing a noise component included in a measurement signal due to optical path characteristics by subtracting a preliminarily acquired reference signal from the measurement signal in computation apparatus 130.

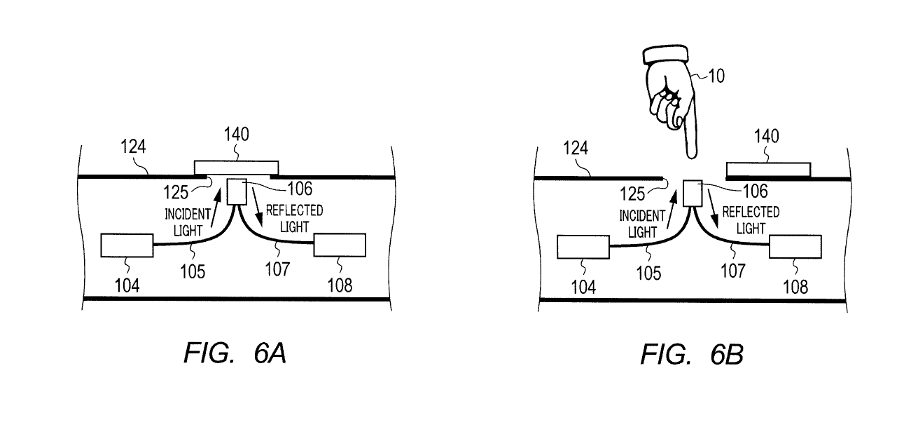

As illustrated in FIG. 6A, when obtaining a reference signal, reflection member 140 is moved to a position where reflection member 140 faces an end of measurement probe 106, and reflection member 140 reflects light emitted from measurement probe 106 toward measurement probe 106. In contrast, as illustrated in FIG. 6B, when obtaining a measurement signal, reflection member 140 is moved out from the position where reflection member 140 faces the end of measurement probe 106. Although not illustrated in FIG. 6 and FIG. 1, it suffices to provide a slide mechanism such as a VCM and a stepping motor for the purpose of moving reflection member 140, for example.

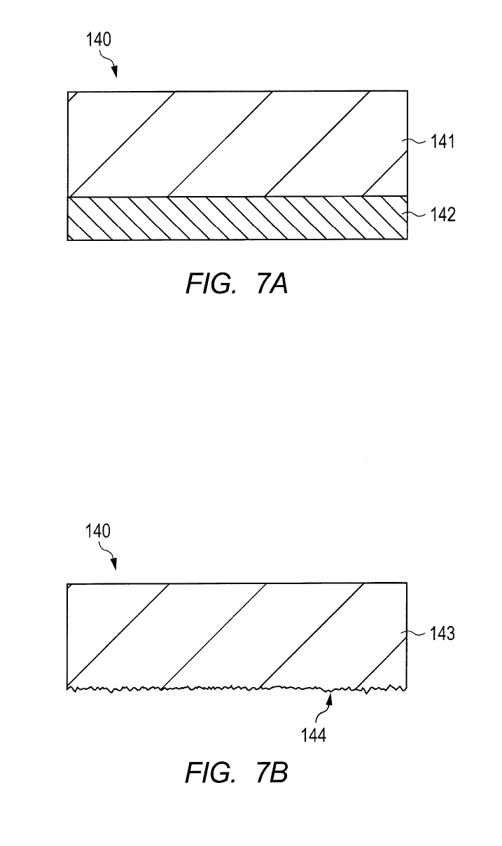

FIG. 7A and FIG. 7B are sectional views illustrating exemplary configurations of reflection member 140. FIG. 7A illustrates an example in which main body 141 is composed of a resin or the like, and metal film 142 is formed by plating or deposition as a reflecting surface. FIG. 7B illustrates an example in which main body 143 is composed of aluminum, stainless-steel or the like, and diffusion reflection surface 144 is formed by forming irregularity such as a satin finished surface as a reflecting surface. Diffusion reflection surface 144 has a roughness approximating a reflectance of skin surface. In this manner, noise of skin surface can be artificially included in a reference signal.

With reflection member 140 of the present embodiment, the following effects can be achieved.

(i) Since an optical path for obtaining a measurement signal and an optical path for obtaining a reference signal are used in common, the device configuration can be simplified and downsized in comparison with the case where the paths are separately provided. In addition, since a reference signal of the optical path same as that of the measurement signal can be obtained, the calibration accuracy can be improved.

(ii) Since the reflecting surface of reflection member 140 is composed of diffusion reflection surface 144 (FIG. 7B) having a reflectance which is close to that of skin surface, the noise of skin surface can be removed as well as the noise of the optical path when a reference signal is subtracted from a measurement signal by calibration, and thus the calibration accuracy can be improved.

(iii) When measurement is not performed, reflection member 140 is moved to a position where opening 125 formed at a position corresponding to measurement probe 106 is closed, and thus intrusion of dust into the optical system can be prevented. That is, reflection member 140 also functions as a closure which closes opening 125, as well as the function of acquiring a reference signal. As a result, in comparison with the case where a dedicated closure is provided, the number of components can be reduced.

As described above, according to the present embodiment, reflection light from subject 10 is split with use of rotation diffraction grid 110, and consequently the number of components of the optical system and the required space can be reduced. In addition, since reflection member 140 which reflects the light emitted from measurement probe 106 and emits the light toward measurement probe 106 is provided in place of subject 10, the optical path for obtaining a measurement signal and the optical path for obtaining a reference signal can be shared. Thus, the required space can be reduced and the calibration accuracy can be improved. As a result, without reducing the measurement accuracy, the optical system and the optical system for the reference signal can be particularly downsized. That is, without reducing the measurement accuracy, biological information measurement device 100 having a small device configuration can be achieved.

In addition, since rotation diffraction grid 110 is achieved by forming a diffraction grid in a MEMS mirror, small-sized rotation diffraction grid 110 can be achieved at a low cost in comparison with the case where rotation diffraction grid 110 is achieved by attaching a diffraction grid to an actuator such as a galvanometer.

A MEMS mirror can be readily produced by a so-called wafer process, and therefore can be produced at a low cost. Further, since diffraction grid 111 can be readily formed by directly forming diffraction grid 111 on a MEMS mirror by a wafer process, increase of cost can be suppressed. In addition, since a diffraction grid is directly formed on a mirror, assembling is unnecessary. Diffraction grid 111 may be formed through a process different from the manufacturing process of the MEMS mirror and may be bonded to the MEMS mirror.

While the first optical path which guides the light emitted from light source 101 to the measurement target and the second optical path which guides the reflection light from the measurement target are composed using optical fibers 105 and 107 in the above-described embodiment, the present invention is not limited to this, and the paths may be composed of a space optical system without using optical fibers 105 and 107.

While the biological information measurement device of the embodiment of the present invention is used for measurement of blood glucose level in the above-described embodiment, the biological information measurement device of the embodiment of the present invention may be used for measurement of biological information other than blood glucose level. For example, by generating an ultraviolet ray having a wavelength of 300 to 400 [μm] in light source 101 and by irradiating subject 10 with the ultraviolet ray, the state of the skin surface of subject 10 can be measured.

It should be understood by those skilled in the art that various modifications, combinations, sub-combinations and alterations may occur depending on design requirements and other factors in so far as they are within the scope of the appended claims or the equivalents thereof. While the invention made by the present inventor has been specifically described based on the preferred embodiments, it is not intended to limit the present invention to the above-mentioned preferred embodiments but the present invention may be further modified within the scope and spirit of the invention defined by the appended claims.

The disclosure of the specification, drawings, and abstract in Japanese Patent Application No. 2013-272964 filed on Dec. 27, 2013 is incorporated herein by reference in its entirety.

### INDUSTRIAL APPLICABILITY

The present invention is applicable to a biological information measurement device which noninvasively measures biological information.

### REFERENCE SIGNS LIST

10 Subject
100 Biological information measurement device
101 Light source
102 Pin hole
103 Condenser lens
104 Light incidence body
105 Light emission side optical fiber
106 Measurement probe
107 Light reception side optical fiber
108 Light emission body
109 Lens system
110 Rotation diffraction grid
111 Diffraction grid
121 Slit
122 Photodetector (PD)
123 Analog digital conversion circuit (AD conversion)
124 Case
125 Opening
130 Computation apparatus
140 Reflection member
141, 143 Main body
142 Metal film
144 Diffusion reflection surface
200 MEMS device
201 Driving part
202 Fixation frame
203 Movable frame
204, 205 Beam part
204a, 204b, 205a, 205b Beam

1. A biological information measurement device comprising:
a light source;
a first optical path configured to guide light emitted from the light source to a measurement target;
a second optical path configured to guide reflection light reflected from the measurement target;
a rotation diffraction grid configured to split reflection light guided from the second optical path;
a light receiving element configured to receive split light from the rotation diffraction grid; and
a reflection member configured to reflect incident light from the first optical path and emit the incident light to the second optical path in place of the measurement target.

2. The biological information measurement device according to claim 1 wherein the rotation diffraction grid includes:
a Micro Electro Mechanical System (MEMS) mirror; and
a diffraction grid formed on a mirror surface of the MEMS mirror.

3. The biological information measurement device according to claim 2, wherein the rotation diffraction grid rotates such that an incident angle of the reflection light guided from the second optical path with respect to the mirror surface is changed, and oscillates in a direction perpendicular to the mirror surface.

4. The biological information measurement device according to claim 1, wherein a reflecting surface of the reflection member is processed to have a reflectance approximating skin surface.

5. The biological information measurement device according to claim 1, wherein:
a measurement probe is provided between the first optical path and the second optical path, the measurement probe being configured to emit light from the first optical path in a direction of the measurement target and allow reflection light reflected from the measurement target to enter the second optical path; and
the reflection member is provided at a position where the reflection member reflects light emitted from the measurement probe and allows the light to enter the measurement probe.

6. The biological information measurement device according to claim 1, wherein:
a case in which an optical system of the biological information measurement device is housed is provided with an opening part for irradiating the measurement target with light from the optical system and allowing reflection light from the measurement target to return to the optical system; and
the reflection member sets the opening part to an opening state by moving out to a position shifted from the opening part when measurement is performed, and moves to a position where the reflection member closes the opening part when measurement is not performed.
